(19) United States
(12) Patent Application Publication
(10) Pub. No.: US 2010/0010102 A1
(43) Pub. Date: Jan. 14, 2010

(54) TRIGGERED RELEASE OF DRUGS FROM POLYMER PARTICLES

(75) Inventors: Krishnendu Roy, Austin, TX (US); Prinda Wanakule, Austin, TX (US); Ankur Singh, Austin, TX (US)

Correspondence Address: CHALKER FLORES, LLP, 2711 LBJ FRWY, Suite 1036, DALLAS, TX, 75234, US

(73) Assignee: Board of Regents, The University of Texas System, Austin, TX (US)

(21) Appl. No.: 12/500,230

(22) Filed: Jul. 9, 2009

Related U.S. Application Data
(60) Provisional application No. 61/079,305, filed on Jul. 9, 2008.

(51) Int. Cl. A61K 47/42

(52) U.S. Cl. 514/772.1; 514/773; 514/774

(57) ABSTRACT

The present invention includes compositions and methods for the controlled delivery of active agents, e.g., drugs, based on one or more release triggers found in the environment in which the active agent-loaded particle is located. The composition and methods include a polymer network having a polymer cross-linked by peptides that include one or more proteolytic cleavage sites.

### CROSS-REFERENCE TO RELATED APPLICATIONS

This application claims benefit of U.S. Provisional application Ser. No. 61/079,305 filed on Jul. 9, 2008, which is incorporated herein by reference in its entirety.

### TECHNICAL FIELD OF THE INVENTION

The present invention relates in general to the field of controlled drug delivery, and more particularly, to the controlled, triggered release of drugs from polymer particles.

### STATEMENT OF FEDERALLY FUNDED RESEARCH

None.

### INCORPORATION-BY-REFERENCE OF MATERIALS FILED ON COMPACT DISC

None.

### BACKGROUND OF THE INVENTION

Without limiting the scope of the invention, its background is described in connection with drug delivery.

A controlled release polymer system comprising a therapeutic, diagnostic, prognostic, or prophylactic agent is taught in U.S. Pat. No. 7,550,441 issued to Farokhzad et al. (2009). The '441 patent describes a conjugate that includes a nucleic acid ligand bound to a controlled release polymer system, a pharmaceutical composition that contains the conjugate, and methods of treatment using the conjugate. The nucleic acid ligand that is bound to the controlled release polymer system, binds selectively to a target, such as a cell surface antigen, and thereby delivers the controlled release polymer system to the target.

U.S. Pat. No. 6,632,671 issued to Unger (2003) relates to nanocapsules and methods of preparing these nanocapsules. The present invention includes a method of forming a surfactant micelle and dispersing the surfactant micelle into an aqueous composition having a hydrophilic polymer to form a stabilized dispersion of surfactant micelles. The method further includes mechanically forming droplets of the stabilized dispersion of surfactant micelles, precipitating the hydrophilic polymer to form precipitated nanocapsules, incubating the nanocapsules to reduce a diameter of the nanocapsules, and filtering or centrifuging the nanocapsules.

WIPO Patent Application WO/2007/139854 (John and Vemula, 2007) discloses a controlled delivery of an anti-inflammatory, chemopreventive drug by an enzyme-triggered drug release mechanism via degradation of encapsulated hydrogels. The hydro- and organo- gelators are synthesized in high yields from renewable resources by using a regioselective enzyme catalysis and a known chemopreventive and anti-inflammatory drug, curcumin, is encapsulated in the gel matrix and released by enzyme triggered delivery. The release of the drug occurs at the physiological temperature and control of the drug release rate is achieved by manipulating the enzyme concentration and temperature. The by-products formed after the gel degradation clearly demonstrated the site specificity of degradation of the gelator by enzyme catalysis. The present invention has applications in developing cost effective, controlled drug delivery vehicles from renewable resources, with a potential impact on pharmaceutical research and molecular design and delivery strategies.

U.S. Patent Application No. 2008/0241256 (Kuhn, 2008) describes calcium phosphate nanoparticle active agent conjugates. Specifically, anticancer agent conjugates are prepared which are suitable for targeted active agent delivery to tumor cells and lymphatics for the treatment of cancer and the treatment or prevention of cancer metastasis.

### SUMMARY OF THE INVENTION

The present invention is an improved drug delivery particle that release drug in response to tissue-specific enzymes. The polymer hydrogel networks include a network of crosslinkable polymer and biomolecules that are sensitive to their environment. Drug/Therapeutic Agent will be incorporated into hydrogel network, which may even be a hydrogel network that is reduced to micro- or nanoparticle scale. Upon delivery to targeted tissue, the biomolecule that are sensitive to their environment will trigger release of drug/therapeutic agent.

For example, the present invention may be used in a for oral drug delivery in which the hydrogel particle protects an embedded drug from harsh gastric contents, and the biomolecule will trigger drug release upon reaching the small intestine, thereby increasing bioavailability. In another example, the multi-functional network crosslinkable polymer is provided in order to provide buffering protection against pH changes, as well as increased mucoadhesiveness to promote intestinal absorption.

In one embodiment, the present invention is a composition comprising a polymer; one or more peptides susceptible to proteolytic cleavage that crosslink the polymers to form a polymer-peptide particle; and one or more drugs disposed within the polymer-peptide particle complex. In one aspect, the composition includes one or more peptides that are cleaved by a serine protease, a threonine protease, a cysteine protease, an aspartic acid protease, a metalloprotease or a glutamic acid protease. In one aspect the two or more polymers are selected from a group comprising polysaccharides, proteins, peptides including hyaluronic acid, alginic acid, chitosan, pectins, heparin, gelatin, agarose, collagen and derivatives thereof, photocrosslinkable derivatives, hyaluronic acid derivatized with methacrylate functionalities, synthetic polymers poly(vinyl alcohol), poly(acrylic acid), and poly(methacrylic acid) and derivatives thereof. In a specific aspect the one or more peptides are cleaved by a Trypsin and the polymers are a 4-armed poly(ethylene glycol) acrylate polymers.

In certain aspects, the polymer is biodegradable, biocompatible or both. In another aspect, the peptide further comprises additional peptides amino-, carboxy- or both amino and carboxy-from the cleavage site. In yet another aspect, the peptide comprises multiple protease cleavage sites.

In another embodiment, the present invention includes a method of fabricating a polymer-based drug delivery particle by mixing a polymer with one or more peptides, wherein the peptide is susceptible to proteolytic digestion; and crosslinking the peptides and the polymer into a polymer crosslinked by the peptides to form a polymer network, wherein a drug loaded into the polymer network is released upon exposure to a proteolytic enzyme that cleaves the peptide. In one aspect, the peptide is bonded to the precursors of the polymer network during polymer network formation. In another aspect, the enzyme that cleaves the peptide is selected from a serine protease, a threonine protease, a cysteine protease, an aspartic acid protease, a metalloprotease or a glutamic acid protease. In another aspect, the method further comprises the step of loading one or more drugs into the polymer network. In yet another aspect, the method further comprises forming one or more polymer network coats in which each coat comprise one or more peptides that are susceptible to proteolytic cleavage by different enzymes in a proteolytic cascade.

In a certain aspect of the method the polymer is selected from a group comprising comprising polysaccharides, proteins, peptides including hyaluronic acid, alginic acid, chitosan, pectins, heparin, gelatin, agarose, collagen and derivatives thereof, photocrosslinkable derivatives, hyaluronic acid derivatized with methacrylate functionalities, synthetic polymers poly(vinyl alcohol), poly(acrylic acid), and poly(methacrylic acid) and derivatives thereof. In specific aspects of the method the one or more peptides are cleaved by a Trypsin and the polymers are a 4-armed poly(ethylene glycol) acrylate polymers.

Yet another embodiment of the present invention is a polymer network made by the method of mixing a polymer with one or more peptides, wherein the peptide is susceptible to proteolytic digestion; and crosslinking the peptides and the polymer into a polymer crosslinked by the peptides to form a polymer network, wherein a drug loaded into the polymer network is released upon exposure to a proteolytic enzyme that cleaves the peptide.

In yet another embodiment, the present invention includes a composition that includes one or more particles that deliver one or more active agents, wherein the particles are made of one or more polymers crosslinked with one or more peptides susceptible to cleavage. In one aspect, the active agent comprises one or more therapeutic agents or one or more diagnostic agents. In another aspect, the peptide comprises multiple protease cleavage sites. In yet another aspect the one or more polymers are a 4-armed poly(ethylene glycol) acrylate polymers and the one or more peptides are susceptible to cleavage by a Trypsin

### BRIEF DESCRIPTION OF THE DRAWINGS

For a more complete understanding of the features and advantages of the present invention, reference is now made to the detailed description of the invention along with the accompanying figures and in which:

FIG. 1 shows a Proton NMR of modified KGHGKK (SEQ ID No.: 1) peptide showing successful acrylation (a) in comparison to imidazole groups (b);

FIG. 2 shows a swollen Michael-type addition hydrogel microparticles from nylon mesh mold;

FIG. 3 is a flow chart of a process for use with the present invention;

FIG. 4 is a diagram that shows one example of a release profile of the present invention;

FIGS. 5A to 5C show a time line of a microparticle degradation study. FIG. 5A shows un-swollen microparticles on slide at initial time point, before exposure to trypsin, FIG. 5B shows swollen microparticles on slide after one hour of trypsin exposure. Surface degradation is apparent on each particle, and FIG. 5C shows swollen microparticles on slide after two hours of trypsin exposure, particle degradation is apparent;

FIGS. 6A and 6B show the degradation of bulk peptide hydrogel by trypsin enzyme at t=initial (6A) and t=after 60 minutes (6B);

FIGS. 7A and 7B show the degradation of hydrogel microparticles by trypsin enzyme at time t=initial: control group (7A) and t=trypsin group (7B);

FIGS. 8A and 8B show the degradation of hydrogel microparticles by trypsin enzyme at time t=10 minutes: control group (8A) and t=trypsin group (8B); and

FIGS. 9A and 9B show the degradation of hydrogel microparticles by trypsin enzyme at time t=30 minutes: control group (9A) and t=trypsin group (9B).

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The present invention describes polymer-based micro or nanoparticles that release one or more therapeutic agents at an organ or site or tissue in response to a physiological or pathological stimulus. The invention further describes a method of making such polymer-based drug delivery system. The present invention describes particles that are triggered to release the therapeutic agent in the presence of enzymes, e.g. in the presence of digestive enzymes or enzymes that may be present in a particular diseased tissue or organ. The particle matrix in the present invention primarily comprises polymers that are cross-linked with specific peptides. The therapeutic agents are protected inside the particles during transport and are then released at the desired site in response to a specific stimulus. This allows for increased efficiency of delivery, protection of therapeutic agents, and reduced side-effects due to tissue-specific release. For example: for oral delivery, the therapeutic agent is protected in the stomach from acidic environment and is released in the intestine when the particle encounters specific digestive enzymes, thus increasing bioavailability.

The present disclosure is a first-of a kind invention on enzyme triggered drug release from micro or nanoparticles, especially for oral drug delivery. Although peptide crosslinked hydrogels have been reported in the literature for tissue engineering applications as well as controlled release of drugs, they have not been formulated into microparticles or nanoparticles. In addition the polymers used in the fabrication of these particles have been previously reported by the present inventors and the said polymers offer unique protection to the encapsulated therapeutic agents, especially from the stomach acids.

Biodegradable polymer particles, such as microparticles and nanoparticles such as biodegradable poly(lactide-co-glycolide) (“PLGA”) microparticles and others, are effective delivery vehicles for the controlled release of therapeutic compositions such as polypeptides, proteins, nucleic acids, vaccines, etc. Biodegradable polymer particles are also effective delivery vehicles for the controlled release of contrast and imaging agents in the human body. They also have applications in diagnostic and therapeutic imaging. However, these particles do not possess any tissue or disease-specific triggered release mechanism and drug release is due to diffusion and hydrolysis. In addition these particles do not possess any properties of actively reducing effects of tissue microenvironments that can degrade the drug prematurely, e.g. drug degradation in the acidic environments of the stomach. The present invention relates to both these components and provides means for tissue specific drug release triggered by biomolecules and also provides means for active protection of the drug from harsh environments.

In certain embodiment of the present invention the peptides are selected based on the proteolytic enzyme or protease that cleaves the peptide or peptides that are used to cross-link polymers to retain an active agent. Examples of enzymes and their cognate cleavage sequences may include, e.g., Arg-C proteinases, Asp-N endopeptidase, Asp-N endopeptidase+N-terminal Glu, BNPS-Skatole, Caspase1, Caspase2, Caspase3, Caspase4, Caspase5, Caspase6, Caspase7, Caspase8, Caspase9, Caspase10, Chymotrypsin-high specificity (C-term to [FYW], not before P), Chymotrypsin-low specificity (C-term to [FYWML], not before P), Clostripain (Clostridiopeptidase B), CNBr, Enterokinase, Factor Xa, Glutamyl endopeptidase, GranzymeB, Hydroxylamine, Iodosobenzoic acid, LysC, LysN, NTCB (2-nitro-5-thiocyanobenzoic acid), Pepsin (pH1.3), Pepsin (pH>2), Proline-endopeptidase, Proteinase K, Staphylococcal peptidase I, Thermolysin, Thrombin or Trypsin.

The polymer used to make the particles can be either natural or synthetic but the polymer must have functional groups for crosslinking, e.g., chemical crosslinking or photocrosslinking; or the polymer can be a mixture of two polymers in which one polymer has functional groups for chemical crosslinking and the other polymer has functional groups for photocrosslinking; or the polymer can have functional groups for photocrosslinking only but must also be capable of creating physical crosslinks (by temperature or pH-induced gelation) or ionic crosslinks through ion-gelation; or the polymer can be a mixture of two polymers in which one polymer has functional groups for photocrosslinking and the other polymer is capable of physical or ionic crosslinking.

Examples of functional groups for chemical crosslinking include hydroxyls, carboxyls, aldehydes, thiols, and amines, while examples of functional groups for photocrosslinking include vinyls, acrylates, methacrylates, and acrylamides. The skilled artisan will readily understand that the functional groups may be mixed between the chemical crosslinking functional groups and chemical crosslinking functional groups. Also the types of crosslinking may include combinations of different crosslinking within a group or groups.

Photocrosslinking can also be accomplished by photoactivating “caged” functional groups for chemical crosslinking (e.g., caged thiols such as 2-nitrobenzyl cysteine that can be activated by UV light and then chemically reacted to form disulfide crosslinks). In addition, the photocrosslinking can be patterned by use of a printed photomask, a virtual photomask using a digital micro-mirror array device, or two photon photolithography using a confocal laser device.

The chemical crosslinking agent may selected from aldehydes, epoxides, polyaziridyl compounds, glycidyl ethers, carbodiimides, and divinyl sulphones. Glycidyl ethers include 1,4-butanediol diglycidyl ether, poly(ethylene glycol) diglycidyl ether, and ethylene glycol diglycidyl ether. The crosslinking reactions can be performed at any temperature and pH required for crosslinking but milder temperatures and pH are preferred.

Natural polymers can be selected from the group of polysaccharides, proteins, and peptides including hyaluronic acid, alginic acid, chitosan, pectins, heparin, gelatin, agarose, and collagen and derivatives thereof, particularly photocrosslinkable derivatives such as hyaluronic acid derivatized with methacrylate functionalities. Synthetic polymers can be selected from the group of poly(vinyl alcohol), poly(acrylic acid), and poly(methacrylic acid) and derivatives thereof.

Although many of the examples presented in the specification depict the crosslinking in a specific order, the present invention provides that the order of crosslinking may be reversed in some instances without altering the invention, e.g., photocrosslinking may be performed first followed by chemical crosslinking or chemical crosslinking may be performed first followed by photo crosslinking.

Crosslinkable polymers chosen for application of the technology to oral drug delivery were 4-armed polyethylene glycol sulfhydryl (SunBioUSA, Orinda, Calif.) and Protasan UP CL113 chitosan chloride salt or any chitosan (Novamatrix, Norway). Environmentally-sensitive biomolecule crosslinkers were designed and custom synthesized (CHI Scientific, Inc., Maynard, Mass.; Institute for Cellular and Molecular Biology Protein Facility, The University of Texas at Austin, Austin, Tex.).

Design of Environmentally-Sensitive Biomolecule Crosslinker.

For oral drug delivery, biomolecule crosslinkers must be non-reactive to primary gastric enzymes, such as pepsin, but reactive to the major intestinal enzymes, trypsin and chymotrypsin. Human hemoglobin protein (subunits alpha, beta, delta, epsilon, gamma-1, gamma-2, mu, theta-1, and zeta) sequences were chosen for mammalian compatibility, computationally cleaved with pepsin, and residual sequence fragments evaluated for reactivity with trypsin and chymotrypsin. Optimization and hydrophilicity (Hopp-Woods Scale) analyses of these fragments then identified 5-7 chain amino acid sequences with maximum enzyme cleavage potential and highly hydrophilic character. The two peptide sequences chosen from the analysis were Lys-Gly-His-Gly-Lys-Lys (KGHGKK) (SEQ ID No.: 1) and Glu-Val-Arg-Ala-His-Gly-Lys (QVRAHGK) (SEQ ID No.: 2).

Functionalization of Crosslinkable Polymer and Biomolecule Crosslinker.

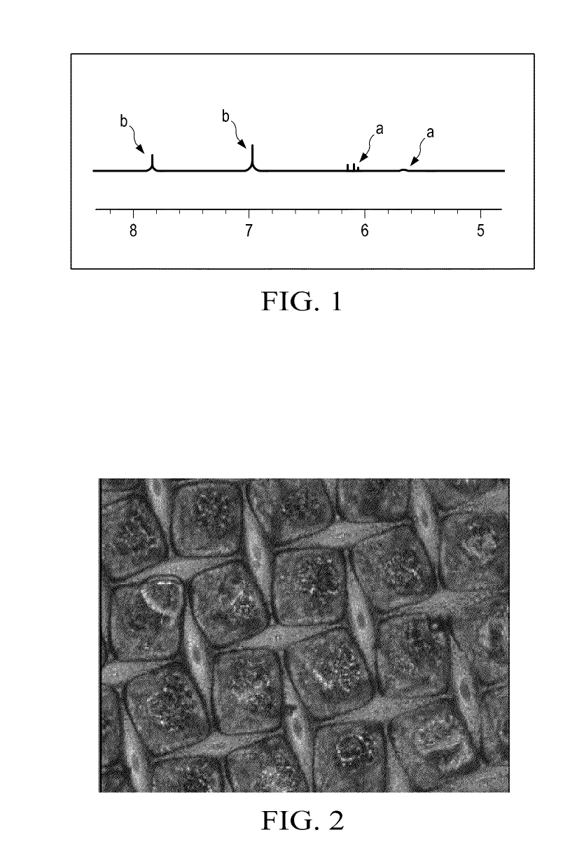

Chitosan was 50% modified with imidazole acetic acid groups, to provide buffering against pH changes and improved solubility, and 50% modified with sulfhydryl groups, to provide functionality for creation of the hydrogel network, using methods previously reported^{1,2}. The biomolecule crosslinkers were modified with a minimum of two acrylate groups per molecule of peptide for reactivity using methods previously described³. Successful modification was verified by proton NMR, as shown in FIG. 1. FIG. 1 shows a Proton NMR of modified KGHGKK (SEQ ID No.: 1) peptide showing successful acrylation (a) in comparison to imidazole groups (b).

Preparation of Hydrogel Network.

Crosslinked hydrogel networks were created by Michael-type addition reaction between the suflhydryl-functionalized crosslinkable polymer and acrylate-functionalized biomolecule crosslinker. Briefly, a solution of 2.5% final (w/v) crosslinkable polymer in IM PBS buffer at pH 7.8, and a corresponding % (w/v) solution for a 1:1 sulfhydryl:acrylate molar ratio of biomolecule crosslinker in IM PBS at pH 7.8 were combined at room temperature and lightly vortexed. The combined solution was then placed in a water bath at 37 degrees Celsius and allowed to cure for 30 minutes.

Methods for Creation of Microparticles.

1. Grinding and Milling. Hydrogels were flash-frozen in liquid nitrogen for 2-3 minutes, then immediately ground using milling processes or mortar and pestle with sieving. Resulting particles were then be lyophilized for storage stability. Particle size distribution and morphology of both dried and swollen particles were analyzed using SEM.

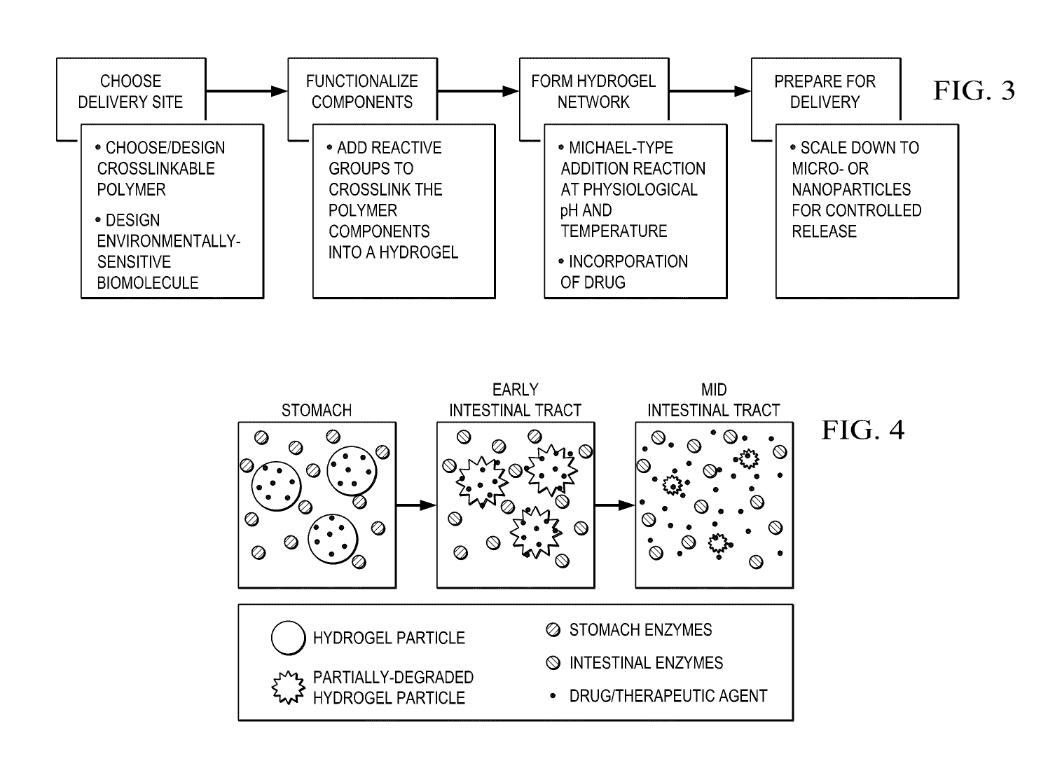

2. Size and Shape-Specific Molding. Hydrogel solutions were poured directly into various molds, such as a PDMS mold created from a lithography template, or over a pore size-specific nylon mesh mold, to create size and shape-specific particles for possible intravenous administration. The solution inside the mold was then cured in an incubator or water bath at 37° C. for hydrogel particle formation. FIG. 2 shows 64 μm-sized swollen hydrogel microparticles (PEG-4-SH and PEG-DA crosslinked by Michael-type addition) created using the nylon mesh mold. FIG. 3 shows a flow chart of the selection process of the present invention for the creation, selection and completion of the hydrogel networks of the present invention.

3. Ultrasound Sonication. A probe sonicator was used to subject samples to sufficiently high shearing stresses for creation of microparticles from a bulk hydrogel block. Resulting hydrogel microparticles were then lyophilized for storage stability.

FIG. 4 is a diagram that shows one example of a release profile of the present invention in which the hydrogel particles are shown loaded with drug(s) in a stomach environment. As the particles enter the early intestinal tract, the outer portion of the hydrogel network of the present invention has undergone a first transformation. As the particles enter the mid intestinal tract and are exposed to different conditions, e.g., pH, salts, enzymes and the like, the particles increase their delivery of the drug(s) to the mid intestinal tract. As the skilled artisan will recognize, the type of peptide polymer linker can be selected to have one or more release profiles, interactions with its environment surrounding the particle (in this example the intestinal wall), location for release, release kinetics, release of additional factors or drugs and the like.

Microparticle Degradation Study.

To demonstrate enzyme-responsive degradation of novel peptide-crosslinked polymer microparticles, 64 mm microparticles of polyethylene glycol tetra-sulfhydryl and the QVRAHGK (SEQ ID No.: 2) diacrylate peptide crosslinker, were created and affixed to a microscope slide using the nylon mesh press method, as described in methods section. The particles were then exposed to trypsin digestion enzyme for two hours, and degradation was visually observed using an optical microscope.

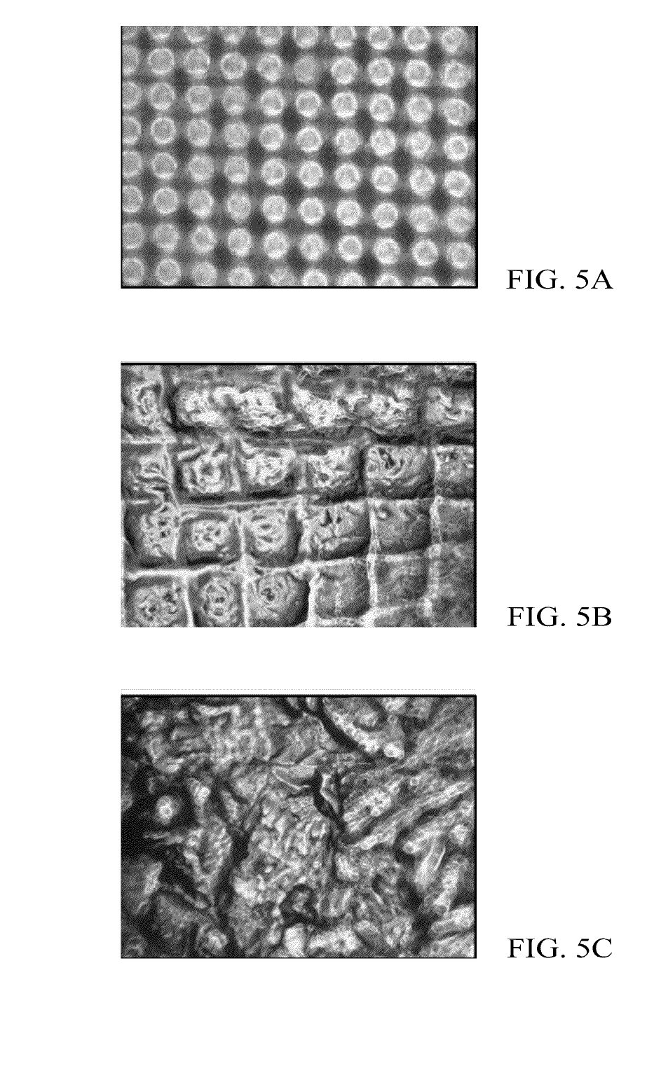

FIG. 5A to 5C show a time line of a microparticle degradation study. FIG. 5A shows un-swollen microparticles on slide at initial time point, before exposure to trypsin, FIG. 5B shows swollen microparticles on slide after one hour of trypsin exposure. Surface degradation is apparent on each particle, and FIG. 5C shows swollen microparticles on slide after two hours of trypsin exposure. Particle degradation is apparent.

Hydrogel Degradation Study

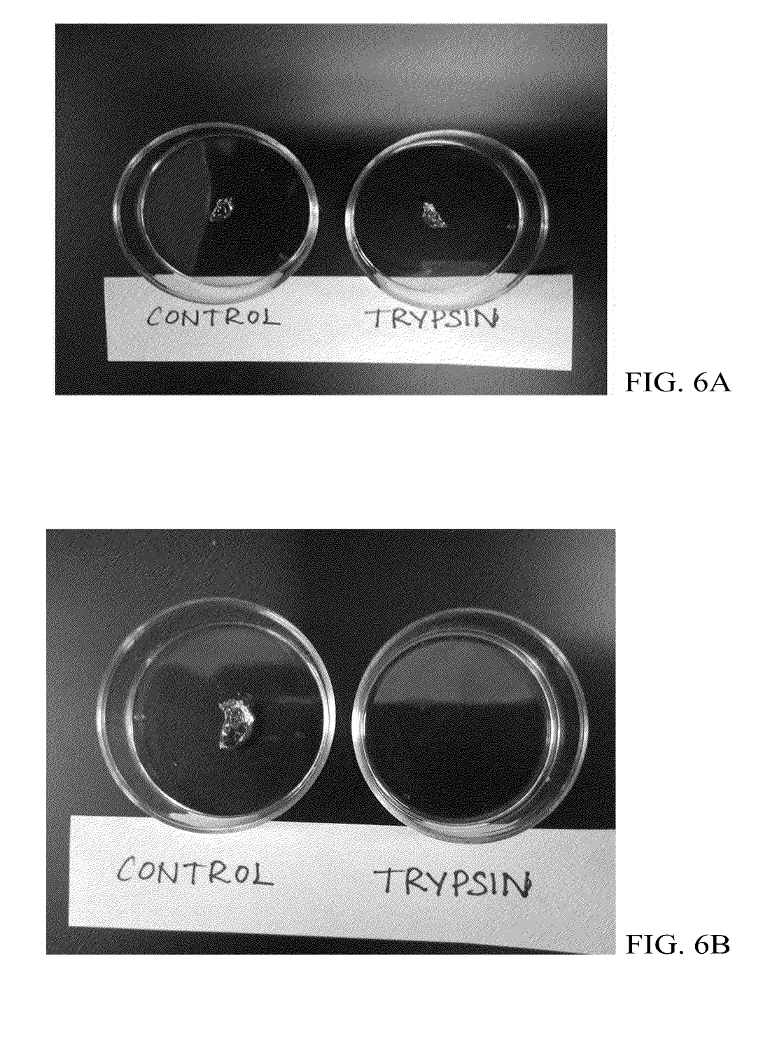

To demonstrate the enzyme-responsive degradation of hydrogel comprising a peptide, a hydrogel was formed via Michael's addition reaction of 4-armed poly(ethylene glycol) acrylate with a peptide (peptide sequence (CGRGGC) (SEQ. ID. No.: 3) at 37° C. in pH 8.15 triethanolamine (TEA) buffer (50% w/v). A single gel was formed and it was halved for each of the two study groups. The control group (gel) was placed in 3 ml of 1× Phosphate Buffered Saline (PBS) at pH 7.4 in a 37° C. incubator with shaking and the Trypsin group (gel) was placed in 3 ml of 20 μg/ml Trypsin in 10% 1 mM HCl, 90% 40 mM NH₄HCO₃ (standard trypsin in-gel digest solution) in a 37° C. incubator with shaking. Images taken with the solution removed. The images are shown in FIGS. 6A (initial time point) and 6B (after 60 minutes).

The results of the study demonstrate the responsiveness of the hydrogel comprising a peptide to the enzyme, Trypsin. The hydrogel in the control group shows no degradation after 60 minutes in PBS, whereas the hydrogel in the Trypsin group has been completely degraded after 60 minutes of enzyme exposure. The study indicates the ability to use such hydrogels for the enzyme-triggered release of therapeutic agents embedded within the hydrogel network, as has not been demonstrated previously for the delivery of therapeutic agents.

Hydrogel Microparticle Degradation Study

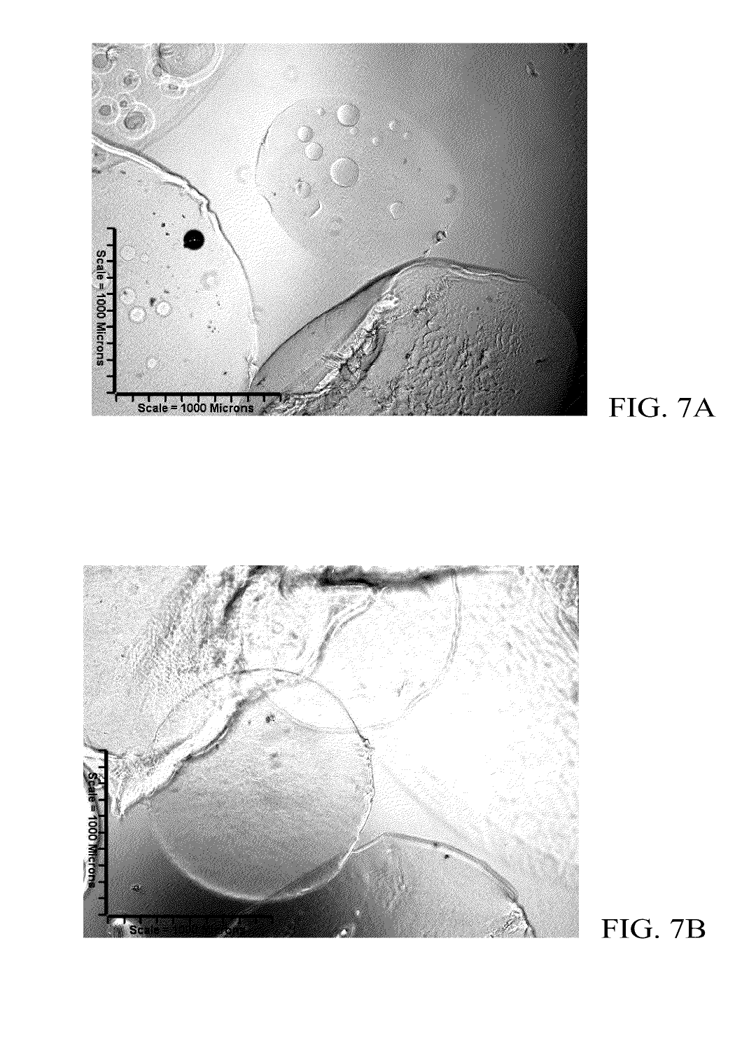

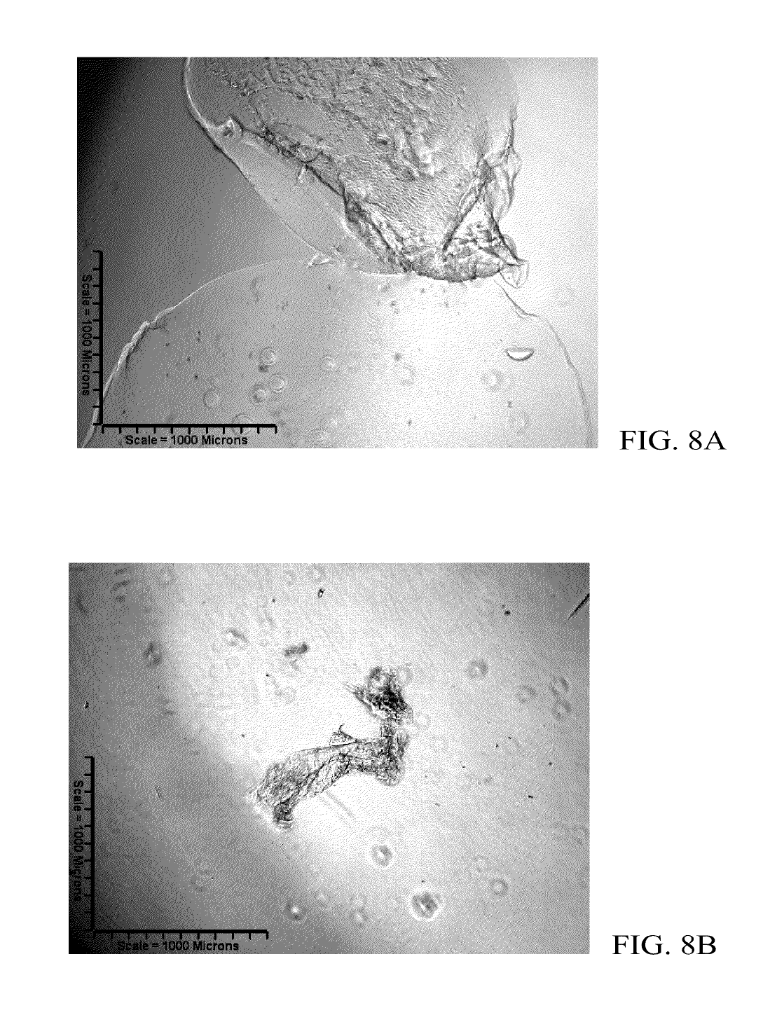

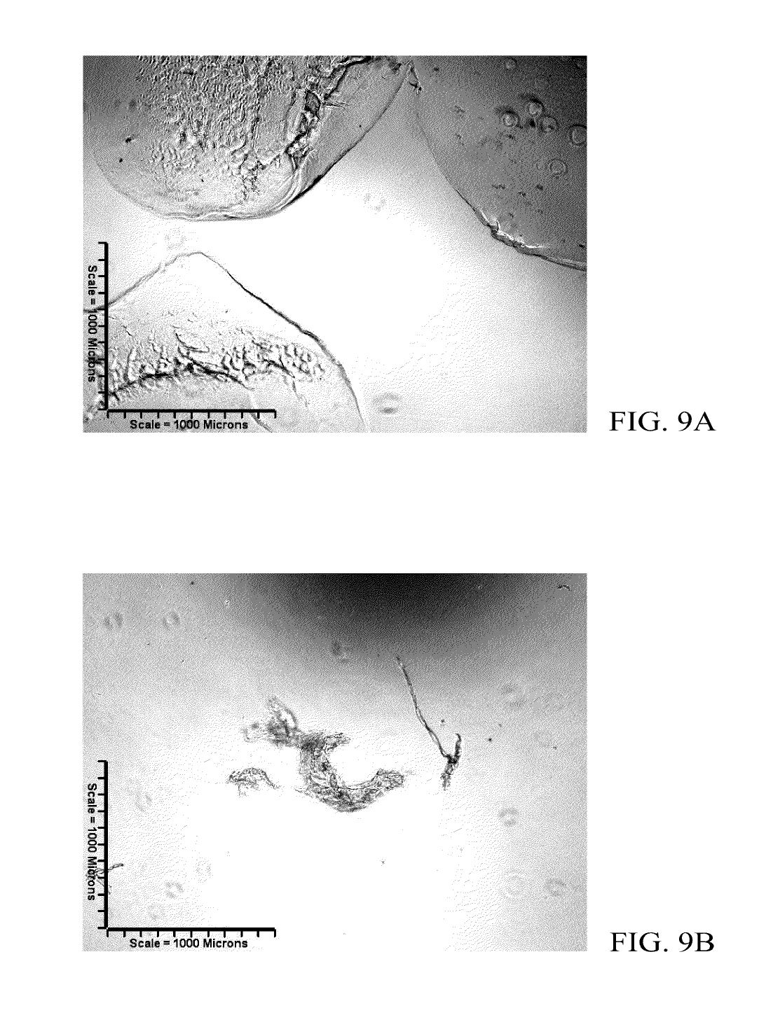

To demonstrate the enzyme-responsive degradation study of a hydrogel microparticle, a hydrogel of 4-armed poly(ethylene glycol) acrylate and a peptide (peptide sequence (CGRGGC) (SEQ. ID. No.: 3) was formed via Michael's addition reaction, at 37° C. in pH 8.15 triethanolamine (TEA) buffer (40% w/v). The pre-hydrogel solution was nebulized, reacted, dried overnight, harvested, and swollen for 1 hour prior to experiment. The experimental design comprised of two groups: (i) Control Group: placed in 1.5 ml of 10% 1 mM HCl, 90% 40 mM NH₄HCO₃ in a 37° C. incubator with shaking and (ii) Trypsin Group: placed in 1.5 ml of 20 μg/ml Trypsin in 10% 1 mM HCl, 90% 40 mM NH₄HCO₃ (standard trypsin in-gel digest solution) in a 37° C. incubator with shaking. Images obtained for the control groups at time t=initial, 10 mins, and 30 mins are shown in FIGS. 7A, 8A, and 9A, respectively. Images obtained for the control groups at time t=initial, 10 mins, and 30 mins are shown in FIGS. 7B, 8B, and 9B, respectively.

The results of the study demonstrate the ability to form microparticles using the proposed Michael's addition reaction, as well as the responsiveness of the hydrogel microparticles comprising a peptide to the enzyme, Trypsin. The hydrogel microparticles in the control group show no degradation after 30 minutes in PBS, whereas the hydrogel microparticles in the Trypsin group show degradation after 30 minutes of enzyme exposure. The study indicates the ability to use such hydrogel microparticles for the enzyme-triggered release of therapeutic agents embedded within the hydrogel network, as has not been demonstrated previously for the delivery of therapeutic agents.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

U.S. Pat. No. 7,550,441: Controlled release polymer nanoparticle containing bound nucleic acid ligand for targeting.

U.S. Pat. No. 6,632, 671: Nanoparticle encapsulation system and method.

WIPO Patent Application WO/2007/139854: Method for preparing hydro/organo gelators from disaccharide sugars by biocatalysis and their use in enzyme-triggered drug delivery.

U.S. Patent Application No. 2008/0241256: Targeted active agent delivery system based on calcium phosphate nanoparticles

Ghosn, B.; Kasturi, S. P.; Roy, K., Enhancing Polysaccharide-Mediated Delivery of Nucleic Acids Through Functionalization with Secondary and Tertiary Amines. Current topics in medicinal chemistry 2008, 8, (4), p 331-340.

Lee, D.; Zhang, W.; Shirley, S.; Kong, X.; Hellerman, G., Thiolated Chitosan/DNA Nanocomplexes Exhibit Enhanced and Sustained Gene Delivery. Pharmaceutical Research 2007, 24, (1), p 157-167.

Hem, D. L.; Hubbell, J. A., Incorporation of Adhesion Peptides into Nonadhesive Hydrogels Useful for Tissue Resurfacing. Journal of Biomedical Materials Research 1998, p 266-276.

What is claimed is:

1. A composition comprising:
two or more polymer particles;
one or more peptides susceptible to proteolytic cleavage that crosslink the particles to form a polymer-particle complex; and
one or more drugs disposed within the peptide-particle complex.

2. The composition of claim 1, wherein the one or more peptides are cleaved by a serine protease, a threonine protease, a cysteine protease, an aspartic acid protease, a metalloprotease or a glutamic acid protease.

3. The composition of claim 1, wherein the two or more polymers are selected from a group comprising polysaccharides, proteins, peptides including hyaluronic acid, alginic acid, chitosan, pectins, heparin, gelatin, agarose, collagen and derivatives thereof, photocrosslinkable derivatives, hyaluronic acid derivatized with methacrylate functionalities, synthetic polymers poly(vinyl alcohol), poly(acrylic acid), and poly(methacrylic acid) and derivatives thereof.

4. The composition of claim 1, wherein the one or more peptides are cleaved by a Trypsin.

5. The composition of claim 1, wherein the polymers are a 4-armed poly(ethylene glycol) acrylate.

6. The composition of claim 1, wherein the polymer is biodegradable.

7. The composition of claim 1, wherein the polymer is biocompatible.

8. The composition of claim 1, wherein the peptide further comprises additional peptides amino-, carboxy- or both amino and carboxy-from the cleavage site.

9. The composition of claim 1, wherein the peptide comprises multiple protease cleavage sites.

10. A method of fabricating a polymer-based drug delivery particle comprising mixing a polymer with one or more peptides, wherein the peptide is susceptible to proteolytic digestion; and crosslinking the peptides and the polymer into a polymer crosslinked by the peptides to form a polymer network, wherein a drug loaded into the polymer network is released upon exposure to a proteolytic enzyme that cleaves the peptide.

11. The method of claim 10, wherein the peptide is bonded to the precursors of the polymer prior to polymer formation.

12. The method of claim 10, wherein the polymer is selected from a group comprising comprising polysaccharides, proteins, peptides including hyaluronic acid, alginic acid, chitosan, pectins, heparin, gelatin, agarose, collagen and derivatives thereof, photocrosslinkable derivatives, hyaluronic acid derivatized with methacrylate functionalities, synthetic polymers poly(vinyl alcohol), poly(acrylic acid), and poly(methacrylic acid) and derivatives thereof.

13. The method of claim 10, wherein the enzyme is selected from a serine protease, a threonine protease, a cysteine protease, an aspartic acid protease, a metalloprotease or a glutamic acid protease.

14. The method of claim 10, wherein the polymer is 4-armed poly(ethylene glycol) acrylate.

15. The method of claim 10, wherein the enzyme is trypsin.

16. The method of claim 10, further comprising the step of loading one or more drugs into the polymer network.

17. The method of claim 10, further comprising forming one or more polymer network coats that each comprise one or more peptides that are susceptible to proteolytic cleavage by different enzymes in a proteolytic cascade.

18. A polymer network made by the method of mixing a polymer with one or more peptides, wherein the peptide is susceptible to proteolytic digestion; and crosslinking the peptides and the polymer into a polymer crosslinked by the peptides to form a polymer network, wherein a drug loaded into the polymer network is released upon exposure to a proteolytic enzyme that cleaves the peptide.

19. The polymer network of claim 18, wherein the polymer is biodegradable.

20. The polymer network of claim 18, wherein the polymer is biocompatible.

21. The polymer network of claim 18, wherein the peptide further comprises additional peptides amino-, carboxy- or both amino and carboxy-from the cleavage site.

22. The polymer network of claim 18, wherein the peptide comprises multiple protease cleavage sites.

23. The polymer network of claim 18, wherein the polymer network further comprises a proteolytic enzyme activator.

24. A composition comprising:
one or more particles that deliver one or more active agents, wherein the particles are made of one or more polymers crosslinked with one or more peptides susceptible to cleavage.

25. The composition of claim 24, wherein the active agent comprises one or more therapeutic agents or one or more diagnostic agents.

26. The composition of claim 24, wherein the peptide comprises multiple protease cleavage sites.

27. The composition of claim 24, wherein the one or more polymers are a 4-armed poly(ethylene glycol) acrylate polymers.

28. The composition of claim 24, wherein the one or more peptides are susceptible to cleavage by a Trypsin.
